(19) United States
(12) Patent Application Publication
(10) Pub. No.: US 2026/0308527 A1
(43) Pub. Date: Oct. 8, 2026

(54) Protocol for maturation of 3D engineered heart tissue

(71) Applicant: Regents of the University of Minnesota, Minneapolis, MN (US)

(72) Inventors: Bhairab N. Singh, Circle Pines, MN (US); Mohammad Shameem, Circle Pines, MN (US); Ahmed M. Sharara, North Oaks, MN (US)

(21) Appl. No.: 19/642,063

(22) Filed: Apr. 8, 2026

Related U.S. Application Data
(60) Provisional application No. 63/785,225, filed on Apr. 8, 2025.

(51) Int. Cl. C12N 5/077

(52) U.S. Cl. CPC C12N 5/0657; C12N 2501/41; C12N 2506/45; C12N 2513/00

(57) ABSTRACT

A method and system for promoting maturation of cardiomyocytes, comprising contacting cardiomyocytes with an agent that modulates GLI signaling, wherein the modulation of GLI signaling induces maturation of the cardiomyocytes, wherein in the cardiomyocytes are human induced pluripotent stem cell-derived cardiomyocytes (hiPSC-CMs) and wherein the agent is GANT61.

### CROSS-REFERENCE TO RELATED APPLICATION

This application claims priority to and the benefit of U.S. provisional application Ser. No. 63/785,225, filed Apr. 8, 2025, the contents of which are incorporated herein in their entirety.

### BACKGROUND

Human induced pluripotent stem cell-derived cardiomyocytes (hiPSC-CMs) provide a scalable platform for cardiovascular research but remain structurally and functionally immature, thereby limiting their utility for studying cardiac biology, disease pathogenesis, and clinical translation. Developmental signaling pathways that coordinate cardiomyocyte maturation remain incompletely understood.

Cardiomyocyte (CM) maturation is a complex process involving structural, functional, and electrophysiological remodeling as CMs transition from the fetal to adult stage (Karbassi et al. 2020, Feyen et al., 2020, Guo et al. 2020). This transition is tightly governed by highly coordinated transcriptional and signaling networks (Guo et al. and Ronaldson-Bouchard, et al. 2018). Defining the molecular underpinnings of CM maturation is essential for fully exploiting the potential of human induced pluripotent stem cell-derived cardiomyocytes (hiPSC-CMs) in cardiac regeneration, disease modeling, and drug discovery (Louch et al., 2015, Lewis-Israeli et al. 2021).

Numerous strategies have been tested to promote hiPSC-CM maturation. For example, long-term culture (Lundy et al, 2013), media formulations (Feyen et al. 2020), electrical stimulation (Chen et al. 2013), hydrodynamic conditioning (Huang et al. 2020), and mechanical stimuli (Ronaldson-Bouchard 2018) have been shown to induce hiPSC-CM maturation. Others have used a combinatorial approach involving small molecules such as thyroid hormone, dexamethasone, and insulin-like growth factor-1 to improve hiPSC-CM maturity (Huang 2020, Wange et al, 2021). Similarly, 3D-engineered heart tissue (EHT) approaches with progressive electrical conditioning have produced CMs with organized ultrastructure, t-tubules, oxidative metabolism, and positive force-frequency relationships. These methods have been shown to improve the expression of functional genes, increase sarcomere length, and improve contractile function in hiPSC-CMs. While these strategies improve functional gene expression, sarcomere organization, and contractile performance, many rely on complex platforms or extended culture periods, rather than recapitulating the intrinsic developmental maturation program. Methods based on developmental cues may better mimic in vivo CM maturation dynamics and preserve proper tissue organization.

Metabolically, immature CMs primarily rely on glycolysis, while the adult mature CMs predominantly utilize fatty acid oxidation (FAO) for ATP production. This metabolic switch is accompanied by increased mitochondrial biogenesis (Ramachandra et al. 2018), via the activation of Peroxisome Proliferator-Activated Receptor Gamma Coactivator 1α (PGC1α) regulated pathways. PGC1α is a central regulator of mitochondrial biogenesis and energy metabolism in CMs (Ventura-Clapier et al. 2008). PGC1α interacts with transcription factors such as nuclear respiratory factor 1 (NRF1) and mitochondrial transcription factor A (TFAM) to control genes governing mitochondrial biogenesis and energy metabolism (Halling et al. 2020). Knockout of PGC1α results in reduced mitochondrial biogenesis with impaired oxidative phosphorylation and heart failure (Lai et al. 2018). These findings underscore the critical role of PGC1α in regulating mitochondrial dynamics in the heart. While the functions of PGC1α in regulating mitochondrial biology are defined, its interactions with other signaling pathways in CM maturation remain unclear.

Previous studies have demonstrated that Hedgehog (HH)-GLI (glioma-associated oncogene homolog) signaling plays a crucial role in cardiac development and maturation (Waldron et al. 2024). The GLI family of transcription factors (GLI1, GLI2, and GLI3) is a central mediator of the HH signaling pathway, activated downstream of HH ligands binding to the Patched1 (PTCH1)/Smoothened (SMO) receptor complex (Singh et al. 2015). Insights from animal models have shown that Gli1⁻/⁻ mice display no cardiac defects, while Gli1⁻/⁻; Gli2⁺/⁻ compound mutants die at birth, and Gli2⁻/⁻;Gli3⁺/⁻ compound mutants exhibit malformations in the cardiac outflow tract (Park et al. 2000, Bai et al. 2002). Notably, a recent study showed that GLI/HH signaling functions as a developmental clock to control the timing of CM differentiation/maturation, and that GLI1 was sufficient to delay differentiation of cardiac progenitors in vitro (Rowton et al. 2022). These findings support the notion that attenuation of GLI signaling may be a key driver of CM maturation. Elucidating the mechanistic role of GLI factors in this context will deepen the understanding of their function during CM development and maturation.

While state-of-the-art differentiation protocols can provide an unlimited supply of hiPSC-CMs, current methods predominantly yield CMs with fetal-like transcriptional, epigenetic, and metabolic profiles, limiting their fidelity as a testbed for therapeutic discovery. Specifically, immature hiPSC-CMs have less developed contractile apparatus, reduced conduction velocity, lower mitochondrial content, and immature metabolic profiles.

### DESCRIPTION OF THE DRAWINGS

FIG. 1A. Heatmap showing the KEGG pathway analysis of physiological functions using the upregulated transcripts from the bulkRNASeq datasets.

FIG. 1B. KEGG enrichment analysis of molecular pathways following GLI-signaling modulation. Note the enrichment of metabolic pathways, steroid biosynthesis, fatty acid metabolism, PPAR signaling, MAPK signaling in the maturation condition.

FIG. 1C. Schematic representation of MYL2-EGFP knock-in reporter hiPSC line and experimental workflow.

FIGS. 1D and E. Flow cytometric analysis and quantification of EGFP expression in MYL2-EGFP reporter hiPSC-CMs following GANT61 treatment. Quantification involved the analysis of hiPSC-CM (n=30,000 cells per condition) (n=6 samples obtained from 3 independent differentiation of hiPSC-CMs in duplicates). Data are presented as mean±SEM. Statistical significance analysis between the control and GANT61-treated groups were determined using one-way ANOVA with Bonferroni test. ns: non-significant; ***p<0.001.

FIG. 1F. Confocal microscopic immunofluorescence staining of α-Actinin showing sarcomere arrangement in hiPSC-CMs following GANT61 (1.5 μM) treatment for 48 hr. Images were obtained from n=6 replicates from 3 independent differentiation of hiPSC-CMs using an Olympus Fluoview 3000 confocal inverted microscope (Scale bar: 200 μm). Nuclei (blue) were stained with DAPI.

FIGS. 1G-1J. MorphoScript analysis of α-Actinin-stained images of hiPSC-CMs (FIG. 1G) (Scale bar 50 μm), and Fourier analysis (FIG. 1H), Sarcomere alignment and orientation distribution (FIG. 1I), quantification of Sarcomere order (FIG. 1J) upon GANT61 (1.5 μM) treatment for 48 hr (n=6 replicates from 3 independent differentiation of hiPSC-CMs). Quantification involved the analysis of n=~150 hiPSC-CM obtained from 3 independent differentiation of hiPSC-CMs. (FIG. 1K) qPCR analysis of MYH7/MYH6 ratio (n=6 replicates from 3 independent differentiation of hiPSC-CMs). Data are presented as mean±SEM. EDF1 transcripts levels were used for expression normalization. Statistical significance analysis between control and GANT61-treated groups were determined using two-tailed unpaired t-test; ***p 0.001.

FIGS. 2A-2E. Whole-cell patch electrophysiology results: Representative action potential (AP) traces (FIG. 2A). Quantification of AP duration time (FIG. 2B), APD20 (FIG. 2C), APD90 (FIG. 2D), and AP decay Tau (FIG. 2E), in hiPSC-CMs following GANT61 (1.5 μM) treatment for 48 hr (n=5 replicates obtained from 3 independent differentiation of hiPSC-CMs). Data are presented as mean±SEM. Statistical significance analysis between control and GANT61-treated groups were determined using two-tailed unpaired t-test; *p<0.05; *p<0.01.

FIG. 2F. qPCR analysis of KCNJ2 transcript following GANT61 (1.5 μM) treatment for 48 hr (n=6 replicates from 3 independent differentiation of hiPSC-CMs). EDF1 transcripts levels were used for normalization. Data are presented as mean±SEM. Statistical significance analysis between control and GANT61-treated groups were determined using two-tailed unpaired t-test; **p<0.01.

FIG. 2G. Immunofluorescence staining of α-Actinin and CX43 in hiPSC-CMs upon GANT61 (1.5 μM) treatment for 48 hr (n=6 replicates from 3 independent differentiation of hiPSC-CMs) (Scale bar: 200 μm). Nuclei were counterstained with DAPI (blue).

FIG. 2H. Quantitative analysis of mean fluorescence intensity using α-Actinin and CX43 immunostained hiPSC-CM following treatment. Analysis was done using multiple confocal fluorescence microscopic images obtained using Olympus Fluoview 3000 confocal inverted microscope at 40× magnification. Data are presented as mean±SEM. Statistical significance analysis between the control and GANT61-treated groups were determined using two-tailed unpaired t-test; *p<0.05.

FIG. 2I. qPCR analysis of PLN transcript using RNA obtained from control and GANT61-treated hiPSC-CMs. EDF1 transcripts levels were used for normalization. (n=6 replicates from 3 independent differentiation of hiPSC-CMs). Data are presented as mean±SEM. Statistical significance analysis in panels between control and GANT61-treated groups were determined using two-tailed unpaired t-test; **p<0.01.

FIG. 3A. Schematic showing the EHT fabrication strategy, and experimental timeline.

FIGS. 3B and, 3C. Representative whole mount images of EHTs and quantitative analysis of their width following following GANT61 (1.5 μM) treatment for 96 hr relative to control. (Each EHTs images captured using M3000 microscope using the same settings and dimensions were analyzed from 2 different regions of the EHTs). (n=6 for control and treated group, analyzed in duplicate for each EHT). Data are presented as mean±SEM. Statistical significance analysis in panels between the control and GANT61-treated groups were determined using two-tailed unpaired t-test; *p<0.05.

FIG. 3D. Confocal microscopy analysis of the EHT sections stained using α-Actinin (green) and CX43 (red) antibodies. White arrows indicate the cellular orientation. The boxed area is magnified in the right panel. (Scale bar: 200 μm). Note the striated pattern and distinct punctate dots in the GANT61 conditions relative to control EHTs. Nuclei (blue) were stained with DAPI.

FIG. 3E. Quantitative analysis of mean fluorescence intensity of CX43 fluorescence in the control and treated EHTs. Analysis was done using multiple fluorescence images obtained using Olympus Fluoview 3000 confocal inverted microscope at 40× magnification. Data are presented as mean±SEM. Statistical significance analysis in panels between the control and GANT61-treated groups were determined using two-tailed unpaired t-test; ***p<0.001.

FIGS. 3F and 3G. qPCR analysis of PLN (FIG. 3F) and RYR2 (FIG. 3G) transcripts using RNA obtained from control and GANT61-treated EHTs. EDF1 transcripts levels were used for expression normalization. (n=4 samples for control and n=6 samples for the treated group for each EHT). Data are presented as mean±SEM. Statistical significance between control and the GANT61-treated group was determined by a t-test; **p<0.01.

FIG. 4A. Pi-chart showing the enrichment of transcripts related to mitochondrial and metabolic pathway following GANT61 treatment in the neonatal cardiomyocytes relative to other cellular component GO-terms.

FIG. 4B. GCN analysis of the upregulated metabolic cascade genes following GLI modulation. Each small dot indicates a transcript and big dot represents their association with the mitochondrial pathway/function. The lines indicate the involvement of each transcript to multiple mitochondrial pathways.

FIG. 4C. Heatmap illustrating upregulation of PGC1α and PPARα transcripts following GANT61 treatment. The heatmap color scheme key is provided based on the log 2 fold-change, with red indicating up-regulated and blue indicating down-regulated transcripts.

FIGS. 4D-4I. qPCR analysis for mitochondrial biogenesis related factors, PGC1α, MFN2, NRF1, MTND6; and fatty acid oxidation-related transcripts, ACSL3, and CPT1A using RNA isolated from control and GANT61 (1.5 μM, for 48 hr) treated hiPSC-CMs. EDF1 transcripts levels were used for normalization. (n=6 replicates from 3 independent differentiation of hiPSC-CMs). Data are presented as mean±SEM. Statistical significance analysis in panels between the control and GANT61-treated groups were determined using two-tailed unpaired t-test; *p<0.05; **p<0.01; ***p<0.001.

FIGS. 4J and 4K. Mitotracker red staining. FIG. 4J, and quantification of the mean red fluorescence intensity (K) using the hiPSC-CMs in control and GANT61 (1.5 μM, 48 hr duration) treated conditions. The dotted-boxed area is magnified in the right panel. Nuclei (blue) were stained with DAPI. Analysis was done using Olympus Fluoview 3000 confocal inverted microscope at 40× magnification. Data are presented as mean±SEM. Statistical significance analysis in panels between the control and GANT61-treated groups were determined using two-tailed unpaired t-test; *p<0.05.

FIG. 5A. Schematic illustration of the hiPSC-CMs treatment with GANT61 (1.5 μM) in the presence or absence of SR18292 (PGC1α inhibitor 10 μM).

FIGS. 5B-5E. Contractile function analysis including contractility profile (FIG. 5B), beat rate (FIG. 5C), max contraction velocity (FIG. 5D), and max relaxation velocity (FIG. 5E) of hiPSC-CMs following treatment with GANT61 alone or in combination with PGC1α inhibitor (SR18292), (GANT61+SR18292) (n=8, each data point indicate average from 4 different ROI, from 3 independent differentiation of hiPSC-CMs in duplicate well). Statistical significance analysis in panels C-E between the control and GANT61-treated groups were determined using one-way ANOVA with Bonferroni test. **p<0.01; ***p<0.001.

FIGS. 5F and 5G. Flow cytometric analysis (FIG. 5F) and quantification (FIG. 5G) of EGFP expression in MYL2-GFP-hiPSC-CMs obtained from control, GANT61 alone and GANT61+SR18292 condition. Quantification involved the analysis of hiPSC-CM (n=30,000 cells per condition). (n=6 replicates from 3 independent differentiation of hiPSC-CMs). Statistical significance analysis between the control and GANT61-treated groups were determined using one-way ANOVA with Bonferroni test. ns: non-significant; ***p<0.001.

FIG. 5H. Representative confocal microscopic images of hiPSC-CMs immunostained using α-Actinin (green) and CX43 (red) antibodies following treatment with GANT61 alone and GANT61+SR18292. Nuclei (blue) were stained with DAPI. (Scale bar 200 μm). (I) Quantitative analysis of CX43 mean fluorescence intensity (MFI) using the confocal images from hiPSC-CMs in control, GANT61 alone and GANT61+SR18292 treated conditions. Analysis was done using an Olympus Fluoview 3000 confocal inverted microscope at 40× magnification. Data are presented as mean±SEM. Statistical significance between groups were determined using one-way ANOVA with Bonferroni test. ns: non-significant; *p<0.05.

FIG. 6A-6H. qPCR analysis of transcripts associated with Ca²⁺ handling, KCNJ2, PLN (FIG. 6A, FIG. 6B); CM maturation, MYH7/MYH6 ratio (FIG. 6C); mitochondrial biogenesis, PGC1α, MFN2, NRF1 (FIG. 6D-6F); and fatty acid metabolism, ACSL3, CPT1A (FIG. 6G, FIG. 6H), using RNA isolated from control, GANT61 alone and GANT61+SR18292 treated hiPSC-CMs. EDF1 transcripts levels were used for expression normalization. (n=6 replicates from 3 independent differentiation of hiPSC-CMs). Data are shown as mean±SEM. Statistical significance between the control, GANT61 alone and GANT61+SR18292 treated conditions was determined using one-way ANOVA with Bonferroni test. ns: non-significant; *p<0.05; **p<0.01; ***p<0.001. Note the increased expression of these transcripts in the GANT61 alone condition, which expression were reduced in the GANT61+SR18292 treated conditions. ns: non-significant.

FIG. 7. Schematic illustration of GLI-modulation induced maturation via the PGC1α axis in hiPSC-CMs and EHTs. Integrating genomics, cellular, and molecular biology approaches, we identified a mechanistic intersection between GLI-signaling and mitochondrial biogenesis in the regulation of hiPSC-CM maturation. In hiPSC-CMs, GLI modulation enhanced sarcomere organization and alignment, improved cellular anisotropy, and improved electrophysiological maturation. In engineered tissues, GLI modulation improved tissue architecture, sarcomeric organization, intercellular coupling, and calcium-handling gene expression. Mechanistically, transcriptomic and network analyses revealed activation of mitochondrial and fatty acid oxidation pathways centered on PGC1α, accompanied by increased expression of mitochondrial biogenesis and FAO-related genes. We established a functional GLI-PGC1α axis in mediating CM maturation. The schematic was generated using the BioRender tool (biorender.com).

FIG. 8A. GCN plot showing signaling pathways enriched upon GANT61 treatment compared to the control. Small circles denote the transcripts; big circles represent the pathway; and lines refers to the bridge between the transcripts and pathways.

FIG. 8B. Schematic showing the hiPSC-CMs differentiation protocol and experimental outline. (8C, 8D) Morphological analysis of hiPSC-CMs following GANT61 Treatment (n=~100 cells for each condition analyzed from 3 independent batches of hiPSC-CMs differentiation) (Scale bar 100 μm). Note the increase in the rod-like hiPSC-CMs in the GANT61 conditions. Data are presented as mean±SEM. Statistical significance analysis between the control and GANT61-treated groups were determined using two-tailed unpaired t-test; ***p<0.001.

FIGS. 8E, and 8F. qPCR analysis of MYH7 and MYH6 transcripts in hiPSC-CMs following GANT61 treatment (n=6 replicates from 3 independent batches of hiPSC-CMs differentiation). Data were shown as mean±SEM. Statistical significance analysis between the control and GANT61-treated groups were determined using two-tailed unpaired t-test; **p<0.01.

FIG. 9A, 9B. Patch clamp analysis showing action potential (AP) decay time (2A), resting membrane potential (2B) in hiPSC-CMs following treatment with GANT61 (n=5 replicates obtained from 3 independent differentiation of hiPSC-CMs).

FIGS. 9C, and 9D. qPCR analysis of RYR2 and ATP2A2 transcripts using RNA obtained from control and GANT61 treated hiPSC-CMs (n=6 replicates from 3 independent differentiation of hiPSC-CMs). EDF1 transcripts levels were used for expression normalization. Data were shown as mean±SEM. Statistical significance between the control and GANT61-treated groups was determined using two-tailed unpaired t-test; ns: non-significant; *p<0.05; **p<0.01.

FIG. 10A. Schematic showing hiPSC cardiac fibroblast differentiation protocol and timeline.

FIG. 10B. Immunostaining of hiPSC (OCT4; green) and hiPSC-cardiac fibroblast (hiPSC-cFBs) (Vimentin; red). Nuclei (blue) were stained with DAPI. (Scale bar: 100 μm).

FIG. 10C. qPCR analysis of VIMENTIN expression in hiPSC-cFB (n=6 replicates from 3 independent batches of hiPSC-cFB differentiation). Data were shown as mean±SEM. Statistical significance analysis between the control and GANT61-treated groups were determined using two-tailed unpaired t-test; ***p<0.001.

FIG. 10D. Flow cytometry analysis of hiPSC-cFB, following staining using TE7 antibodies. Note the high efficiency of the hiPSC-cFB differentiation.

FIGS. 10E, and 10F. Design and dimensions of the 3D-printed negative mold.

FIG. 10G. Image of casted PDMS mold. (H) EHTs were fabricated in the PDMS mold and allowed to compact and maintained in RPMI/B27+ media.

FIG. 11A. Mitotracker red staining of hiPSC-CMs following treatment with different concentration (5-20 μM) of PGC1α inhibitor (SR18292) treatment. Images were captured at 20× magnification using an EVOS 5000 microscope (Scale bar 100 μm). Note the concentration dependent reduction in the mitochondrial labyrinth in the SR18292 treated hiPSC-CMs.

FIG. 11B. Quantitative analysis of the rod-like morphology of the hiPSC-CMs following SR18292 treatment. Note that SR18292 treatment does not impact the morphology of the early stage hiPSC-CMs. Data were shown as mean±SEM. Statistical significance analysis was determined using one-way ANOVA with Bonferroni test. ns: non-significant.

FIG. 11C. qPCR analysis of BAX transcripts following PGC1α inhibitor treatment (n=6 replicates from 3 independent differentiation of hiPSC-CMs). Data were shown as mean±SEM. Statistical significance analysis between the control and GANT61-treated groups were determined using two-tailed unpaired t-test; ns: non-significant.

FIG. 12A-12C. qPCR analysis for RYR2, MYH7, and MYH6 transcripts following treatment with GANT61 alone or in combination with SR18292 (GANT61+SR18292) (n=6 samples from 3 independent differentiation of hiPSC-CMs in duplicates). Data were shown as mean±SEM. Statistical significance between the control and GANT61-treated groups in the presence or absence of PGC1α inhibitor was determined using one-way ANOVA with Bonferroni test. ns: non-significant; *p<0.05; **p<0.01.

TABLE 1: List of qPCR primers used in this study.

### SUMMARY

As aspect of the present disclosure relates to a method for promoting cardiomyocyte (CM) maturation comprising modulating GLI signaling in engineered heart tissue (EHT), wherein the cardiomyocytes (CMs) in 2D, and 3D engineered tissue, or both and further comprising treating the CMs with GANT61 for maturation of the CMs achieved in a reduced period time. Treating the CMs at an early stage, for a short time, achieves a mature adult-like phenotype, where in the time of treating is about four days for 3D-tissue.

Another aspect of the present disclosure relates to a small molecule for use in promoting cardiomyocyte (CM) maturation at a reduced concentration of the molecule and in a reduced time to mature, the molecule being GANT61.

In any one of the embodiments herein, the CMs are hiPSC-CMs.

Methods described herein include those for promoting and/or inducing maturation of human induced pluripotent stem cell-derived cardiomyocytes, contacting the cardiomyocytes with GANT61 in culture, wherein the GANT61 inhibits GLI signaling and thereby promotes structural, electrophysiological, and metabolic maturation of the cardiomyocytes.

Another aspect of the present disclosure relates to a composition and system for promoting and/or inducing the maturation of CMs, including hiPSC-CMs, the system comprising a supply or quantity of cardiomyocytes derived from pluripotent stem cells and a composition or culture medium comprising a GLI signaling modulator via activating a PGC1α-mediated metabolic maturation pathway. The GLI signaling modulator may comprise GANT61 in one or more embodiments and may be present in the culture medium a concentration of 0.5 μM to 2.0 μM. This is an amount reduced from the prior art and which as discussed herein exhibits improvements in the mature-like hiPSC-CMs.

Aspects described herein include methods for improving and/or enhancing and/or producing mature-like CMs, including hiPSC-CMs which exhibit one or more characteristics including enhanced sarcomere organization, increased sarcomere alignment, enhanced electrophysiological properties, improved calcium handling, reduced spontaneous beat rate, increased contractile force generation or combinations thereof and thus the methods, systems and compositions described herein may be methods, systems or compositions of or for enhancing sarcomere organization, increasing sarcomere alignment, enhancing electrophysiological properties, improving calcium handling, reducing spontaneous beat rate, increasing contractile force generation of hiPSC-CMs or combinations thereof.

Yet another aspect of the present disclosure relates to method for promoting maturation of cardiomyocytes in engineered cardiac tissue, comprising providing engineered cardiac tissue comprising immature cardiomyocytes, for example, 3D engineered heat tissue (3D-EHT) and culturing the CMs in vitro in the presence of an agent that modulates GLI signaling and increases PGC1α expression, thereby producing cardiomyocytes having increased metabolic maturity relative to cardiomyocytes cultured in the absence of the agent. Similarly, these methods can be conducted with 2D tissues in a monolayer.

### DETAILED DESCRIPTION

Described herein are protocols providing short-term (2-4 days) pharmacological modulation of GLI signaling using GANT61 to enhance the maturity of early-stage hiPSC-CMs in a concentration-dependent manner. GANT61 treatment promoted structural, functional, electrophysiological, and metabolic maturation in both 2D monolayer cultures and 3D EHTs. Mechanistically, GLI signaling modulation promoted CM maturation in association with activation of the PGC1α pathway and its downstream effectors, including NRF1 and MFN2. As described herein, GLI signaling is identified as an important modulator of CM maturation and this disclosure establishes a scalable framework for enhancing the physiological relevance of hiPSC-derived cardiac models.

Efficient strategies to promote hiPSC-CM maturation via the modulation of GLI signaling factors in both 2D and 3D-engineered tissue models are discussed herein. Using a MYL2-EGFP knockin reporter line, hiPSC-CMs treated with Gant61, a pan-GLI inhibitor, led to enhanced CM maturation in a dose-dependent manner. Enhanced maturation in the 2D condition was associated with increased anisotropic cell morphology, multinucleation, improved Ca2+ handling, reduced beat rate, and improved electrophysiological properties. In engineered 3D tissue, treatment with GANT61 for 48 hours resulted in aligned cardiomyocytes with organized sarcomeric structures, improved tissue mechanics, and increased force generation. Additionally, after undertaking mechanistic characterization, GLI-modulation promoted CM maturation via the activation of PGC1α cascade. GLI-modulation influenced mitochondrial network dynamics as seen by increased mitochondrial membrane potential. Moreover, qPCR analysis revealed increased expression of mitochondrial biogenesis factors including PGC1α, NRF1, MFN1/2, ASCL3 etc. Finally, the inhibition of PGC1α led to the complete abrogation of CM maturation induced by GLI-modulation. This data demonstrates the mechanistic involvement of GLI signaling factors in cardiomyocyte maturation, highlighting a robust developmental pathway that can be manipulated to induce optimal hiPSC-CM maturation.

As such this disclosure relates to protocols, systems and culture mediums using GLI signaling as a mediator of hiPSC-CMs maturation. Pharmacological modulation of the GLI-signaling pathway induces transcriptional, structural, functional, and metabolic changes with a shift toward a more mature hiPSC-CM phenotype in both 2D and 3D systems. The methods show support for the PGC1α dependent axis, linking GLI modulation to mitochondrial biogenesis and improved CM maturation. These mechanistically informed strategies are provided to enhance maturation-associated programs to promote relevance in disease modeling, drug discovery, and regenerative medicine.

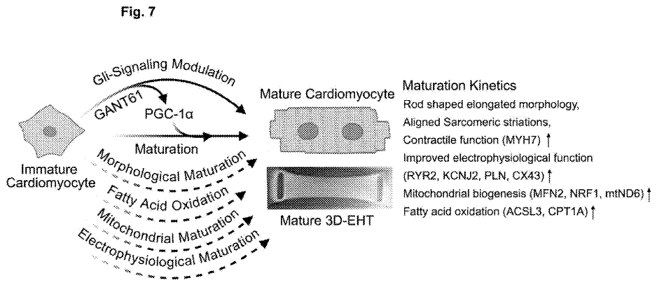

While hiPSC-CMs are described herein with potential for diverse applications, exploiting that potential is dependent upon the maturation status of the hiPSC-CMs. In this disclosure, it is shown that modulation of developmental GLI-signaling promotes the functional, physiological, and metabolic maturation of hiPSC-CMs (see FIG. 7). This approach provides mechanistic insight into developmental control of CM maturation and identifies strategies for improving the therapeutic relevance of hiPSC-CMs. Applicability of these networks in shown in both 2D-monolayer and 3D-engineered tissues and GLI signaling is shown as a tractable target for overcoming barriers to CM maturation.

In one or more embodiments described herein, methods for promoting maturation of cardiomyocytes comprise contacting cardiomyocytes with an agent that modulates GLI signaling, wherein the modulation of GLI signaling induces maturation of the cardiomyocytes, wherein in the cardiomyocytes are human induced pluripotent stem cell-derived cardiomyocytes (hiPSC-CMs). The modulation of GLI signaling comprises inhibition of one or more GLI transcription factors selected from GLI1, GLI2, and GLI3. The agent is a small molecule inhibitor of GLI signaling, and in some examples may comprise GANT61. While GANT61 is discussed in detail below, other small molecule inhibitors of GLI signaling may be used that also capable of activating a PGC1α-mediated metabolic maturation pathway.

In one or more embodiments, the hiPSC-CMs are cultured in a two-dimensional monolayer culture or comprise three-dimensional engineered cardiac tissue (3D-EHT).

The hiPSC-CMs may be contacted with the agent for about 24 hours to about 96 hours. It is contemplated and within the scope of this disclosure that shorter durations, such as greater than about 12 hours, greater than about 18 hours or greater than about 24 hours is sufficient to affect the results discussed herein. Further, longer contact is also contemplated such that contact can be greater than about 36 hours, greater than about 42 hours, greater than about 48 hours, greater than about 72 hours, greater than about 96 hours or greater than about 120 hours or any increment therebetween or thereabove.

The agent may be administered at a concentration of about 0.5 μM to about 2 μM. However, concentrations in the range of about 0.1 μM to about 2.5 μM or greater are within the scope of this disclosure.

Maturation of the cardiomyocytes comprises increasing expression of cardiomyocyte maturation markers and the markers may be selected from MYH7, MYL2, KCNJ2, PLN, RYR2, and CX43. In some embodiments, the agent is selected such that modulation of GLI signaling activates a PGC1α regulatory pathway. Activation of the PGC1α pathway increases expression of genes involved in mitochondrial biogenesis and the gene comprises NRF1, MFN1, MFN2, CPT1A, or ACSL3.

In one or more embodiments, the method for inducing maturation of human induced pluripotent stem cell-derived cardiomyocytes, comprises contacting the cardiomyocytes with GANT61 in culture, wherein the GANT61 inhibits GLI signaling and thereby promotes structural, electrophysiological, and metabolic maturation of the cardiomyocytes. The cardiomyocytes may thus exhibit increased sarcomere alignment following treatment. The cardiomyocytes may thus exhibit increased expression of MYH7 relative to MYH6.

Additional embodiments include a cardiomyocyte maturation culture system, comprising a supply of cardiomyocytes derived from pluripotent stem cells; and a GLI signaling modulator present in culture medium in an amount sufficient to promote cardiomyocyte maturation. For example, in some embodiments the modulator is present in the culture medium at a concentration of 0.5 μM to 2.0 μM. The GLI signaling modulator may be GANT61 and the supply of cardiomyocytes may be human induced pluripotent stem cell-derived cardiomyocytes (hiPSC-CMs) cultured in a 2-dimensional monolayer and/or cardiomyocytes derived from pluripotent stem cells are human induced pluripotent stem cell-derived cardiomyocytes (hiPSC-CMs) are 3-dimensional engineered heart tissue.

As used throughout this disclosure, the term “hiPSC” refers to human induced pluripotent stem cell-derived cardiomyocytes.

As used throughout the disclosure, the term “Cardiomyocytes” refers to the specialized, involuntary muscle cells of the heart's myocardium (wall).

As used throughout the disclosure, the term “GLI factors” refers to GLI transcription factors (e.g., GLI1, GLI2, GLI3), downstream effectors of Hedgehog (Hh) signaling, which are crucial regulators of cardiomyocyte development, balancing proliferation and differentiation. They promote early progenitor proliferation while inhibiting premature maturation.

As used throughout the disclosure, the term “mitochondrial biogenesis” refers to the cellular process of generating new, healthy mitochondria to increase mitochondrial mass and replace damaged ones, thereby increasing the cell's energy capacity. This generally refers to a self-renewal route, the process by which new mitochondria are generated from the ones already existing.

As used throughout the disclosure, the term “maturation” refers to the final stages of differentiation and development where cells, tissues, or organs transform from a generalized, immature state into a more mature form. This process involves structural and biochemical changes that allow the tissue to attain its better functional capacity and structural integrity.

As used throughout the disclosure, the term “3D Engineered Cardiac Tissue” also referred to as 3D Engineered Heart Tissue or “EHT” refers to heart tissue generated through various fabrication methods such as rotational jet spinning, manual seeding in casted molds, bioprinting, and other methods or approaches to fabrication of such tissues known in the field. As discussed herein, one method includes bioprinting tissue and/or living structures formed by the 3D printing of living biological tissues. 3D Tissue engineering models employ the use of 3D matrices to culture cells and create tissues that mimic morphology and function that occur naturally in vivo.

As used throughout the disclosure, the term “Fibroblast” means a type of cell that contributes to the formation of connective tissue.

Despite the emergence of hiPSC-CMs as promising platforms for regenerative medicine, disease modeling, toxicity, and drug screening, their immature phenotype limits their full translational potential. Several approaches including media formulations, biomechanical, chemical, and genetic strategies have been explored to enhance their maturation towards adult-like phenotypes with functional and physiological improvements. However, even the most advanced methodologies remain insufficient in recapitulating the complex physiology, highlighting the need for developmentally informed and mechanistically grounded strategies. Protocols described herein integrated transcriptomics, cellular, and molecular biology approaches to define the intersection between GLI-signaling and mitochondrial biogenesis in the modulation of hiPSC-CM maturation. Pharmacological modulation of GLI signaling promotes morphological, structural, functional, metabolic, and transcriptional features consistent with CM maturation, in association with activation of the PGC1α-mediated programs.

CM maturation is a complex process and involves coordinated morphological, metabolic, and electrophysiological remodeling over time to attain fully mature status. Essentially, CMs undergo morphological, metabolic, and functional changes such as polyploidization, hypertrophy, patterned sarcomeres, t-tubule formation with positive force-frequency coupling. Although these multi-adaptive changes continue to occur even after birth, it is unclear whether they are temporally coordinated or governed by shared regulatory networks. Moreover, signaling factors and their interactions with other regulatory networks involved in CM maturation are poorly understood. Defining these networks is critical for understanding the mechanisms that govern CM development and disease. Increasing evidence emphasizes the role of GLI-signaling in cardiac development.

As described herein, GANT61-mediated modulation of GLI signaling leads to increase in EGFP expression in the MYL2-EGFP-CMs in a concentration-dependent manner, accompanied by enhanced sarcomere ordering and striation compared to control. This was associated with increased expression of cardiac maturation markers, including MYH7, KCNJ2, PLN, RYR2, as well as prominent CX43⁺ puncta at the CM membrane relative to the control. Under GANT61-conditions, the CMs also have rod-shaped morphology and reduced beat rate. Herein a previously undescribed role of GLI-signals in the regulation of CM maturation is disclosed. Given that GLI signaling (particularly GLI2 factors) is downregulated in the postnatal developing heart, we propose that its downregulation represents one of the mechanisms by which CM maturation is augmented.

As discussed herein, GLI modulation is associated with activation of the PGC1α regulatory network in hiPSC-CMs. Previous studies have shown that CMs maintain developmental heterogeneity with variable expression levels of genes responsible for cell growth and contractile function. Further, it is shown that PGC1α signaling was required to regulate genes crucial for hypertrophy and contractility, together with mitochondrial genes. Transcriptomic analysis reveals an upregulation of genes associated with mitochondrial biogenesis and metabolic pathways, including PGC1α and Ppara. PGC1α is a well-established master regulator of mitochondrial biogenesis and cellular energy metabolism. Notably, GLI-modulation significantly increased PGC1α expression and its downstream effectors, resulting in enhanced mitochondrial biogenesis and oxidative metabolism, key features required to meet the elevated energy demands of maturing cardiomyocytes. Importantly, when PGC1α function was inhibited, GLI-modulation failed to induce the expression of maturation markers, mitochondrial biogenesis-related genes, and fatty acid metabolism-associated genes. These findings indicate that PGC1α activity is required for GLI modulation-induced maturation of hiPSC-CMs, supporting a functional GLI-PGC1α axis. It is known that PGC1α signaling remains inactive in hiPSC-CMs, and they are mostly arrested at the late embryonic stage in vitro.

However, since PGC1α signaling is inactive in hiPSC-CMs, factors involved in promoting PGC1α signaling could result in enhanced CM maturation. This is further supported by the lack of structural and functional maturation in mice with CM-specific PGC1α knockout. While the data herein supports the GLI-PGC1α axis in executing the maturation index, it is also acknowledged that, it is not clear whether GLI directly regulates PPARGC1A transcription or acts through intermediate factors. Future studies will be needed to determine the intermediate factors involved in upregulating the PGC1α expression. In the bulk-RNAseq data, increased expression of MEF2, GATA4, etc. was found, all of which are demonstrated to bind to the promoter region of PGC1α. It is possible that GLI-modulation mediates these cascades to regulate PGC1α signaling.

Protocols described herein include modulation of GLI-signals promotes cellular and spatial organization in a 3D-environment. This cellular architecture and structural maturation correlated with improved sarcomeric organization and tissue mechanics. Previously, it was shown that maturation media (MM) containing fatty acids led to enhanced maturation, with better sarcomeric organization, and long-term stability of EHTs. The data herein also supports these findings including stable EHTs, sarcomeric organization, and improved tissue mechanics. This is well correlated with increased expression of genes involved in the structural and physiological functions such as MYH7, PLN, RYR2. Exploring the impact of GANT61 together with maturation media (MM) to further enhance maturation kinetics will be the focus of future studies.

There are multiple developments disclosed herein that are critical to achieving the success described.

Bioinformatic analysis has shown GLI modulation promotes maturation kinetics. Further, Gant61 (small molecule, pan GLI inhibitor) is shown to be a new molecule to promote CM maturation in 3D engineered tissue. Advantageously it has been found that through the disclosed protocols, GANT61 promotes CM maturation at two-fold lower concentration than the previously described.

Usage of GANT61 to promote CM maturation is demonstrated using two independent hiPSC-CM lines. For example, GANT61 treatment for 4-day duration improves 3D tissue alignment with increased cell-cell connectivity and GANT61 treatment for 4-day duration results in functional improvement in 3D tissue including increased force generation, sarcomeric density and orientation.

Further yet, improvement in the maturation process is mediated via increasing mitochondrial biogenesis through the PGC1α axis. Small molecule mediated inhibition of PGC1α abrogates the Gant61 induced cardiomyocyte maturation as also evidenced herein.

### EXPERIMENTAL

### Methods and Materials

hiPSC Culture and hiPSC-CM Differentiation

Two independent hiPSC lines, 1) hiPSC-MLC2v-EGFP and 2) CF-hiPSC lines (reprogrammed from cardiac fibroblasts of a healthy female donor) were used in this study. hiPSC cells were cultured in Matrigel-coated plates using mTeSR1 medium (Stem Cell Technologies, #85850) at 37° C. in a humidified incubator with 5% CO₂. hiPSC-CM differentiation was induced using our established protocol with minor modifications as previously described. Briefly, hiPSCs were washed with 1×DPBS, dissociated by incubation with Accutase solution (Sigma Aldrich, #A6964), and 0.5×10⁶ single cells were seeded in each well of a Matrigel-coated 12-well plate in mTeSR1 medium supplemented with ROCK inhibitor (RI) (10 μM; Y-27632) (Selleck Chemicals, #S1049). After 24 h, media was replaced with fresh mTeSR1 media (2 ml/well) without RI. Upon 90-100% confluency (Day 0), the media was replaced with RPMI/B27 minus insulin (Gibco #A18956-01) containing CHIR99021 (7 μM; glycogen synthase kinase-3 inhibitor) (Sigma Aldrich, #SML1046). On day 2 of differentiation (48 h post CHIR treatment), the media was replaced with RPMI/B27 minus insulin containing IWP2 (7.5 μM; Wnt pathway inhibitor) (Tocris #3533). On day 4 of differentiation, cells were fed with fresh RPMI/B27 minus insulin (2 ml/well). On day 6, the media was replaced with fresh RPMI/B27 containing insulin supplement (RPMI/B27+) (2 ml/well) (Gibco, #17-504-044). On day 11, hiPSC-CMs were washed with 1×DPBS, dissociated with 0.25% trypsin for 16 min at 37° C. and replated at a 1:3 ratio in Matrigel-coated 12 well-plates in RPMI/B27+ media supplemented with RI (10 μM). Between days 13-16, metabolic selection was performed by culturing cells in glucose-free DMEM (Gibco, #11966025) media supplemented with lactate (4 mM). On day 16, cells were washed with 1×DPBS, and maintained in RPMI/B27+ medium with fresh media changes every 3-days and utilized for experiments.

hiPSC-Cardiac Fibroblast (hiPSC-CF) Differentiation

Differentiation of hiPSC-CFs was performed as previously described (Shameem, M. et al. 2025), with minor modifications. Briefly, hiPSCs were maintained in mTeSR1 medium at 37° C. in a humidified incubator with 5% CO₂. At 70-80% confluency, cells were washed with 1×DPBS and dissociated using Accutase solution. Single-cell suspensions were counted using a hemocytometer, and 0.5×10⁶ cells were seeded per well in a Matrigel-coated 12-well plate using mTeSR1 media supplemented with 10 μM RI. After 24 h of plating (Day 0), media was replaced with RPMI/B27 minus insulin containing CHIR99021 (6 μM). On day 1 (24 h post-CHIR), media was replaced with fresh RPMI/B27 minus insulin. On day 3, media was changed to RPMI/B27 minus insulin supplemented with IWP2 (5 μM). After 48 h (Day 5), cells received fresh RPMI/B27 minus insulin. On day 6, cells were dissociated using Accutase solution and seeded as single-cell suspensions onto gelatin-coated plates in LASR medium (DMEM/F12 Advanced, 1.25% GlutaMAX, 60 μg/mL ascorbic acid) supplemented with RI (10 PM). On day 7, media was replaced with LASR containing CHIR99021 (3 μM) for 48 h. Between days 9-12, cells were maintained in LASR media with media changes every 2 days. On day 12, pro-epicardial cells were dissociated with Accutase solution and replated on gelatin-coated 6-well plates in LASR medium containing RI (10 μM). On day 13, media was replaced with fresh LASR media supplemented with 10 ng/mL bFGF (basic fibroblast growth factor) (R&D Systems #233-FB-010) and TGF-β type I receptor ALK5 kinase Inhibitor (0.5 μM; A83-01; MCE #HY-10432), refreshed every 2 days until day 20. On day 20, cells were replated and maintained in complete FibroGRO medium (Millipore Sigma, #SCMF002) in 6-well plates, with fresh media change every 2 days until reaching 70-90% confluency. Differentiated hiPSC-CFs and efficiency were characterized by TE-7 (Fibroblast marker) staining, and purity was confirmed via flow cytometry analysis.

### Fabrication of 3D-Engineered Heart Tissue (EHT)

The EHT fabrication protocol was adapted from a previously described method (Givens et al. 2025, Hosseini et al. 2024). In brief, EHT negative mold was printed using a Stratasys J750 PolyJet 3D Printer with digital acrylonitrile butadiene styrene (ABS). The ABS-negative mold was thoroughly cleaned and treated with silane vapors for 24 h in a vacuum desiccator. Sylgard-184 polydimethylsiloxane (PDMS) (Elsworth Adheisve, #1696742) precursor was mixed with curing agent at a 1:10 mass ratio and cast into the ABS-negative mold. The curing PDMS was degassed for 1 h at room temperature and cured at 60° C. for an overnight period. The resulting PDMS molds were sonicated in 70% ethanol for 30 min and autoclaved at 121° C. for 25 min. The PDMS EHT molds were then transferred to a 12-well plate and treated with 0.5 w/v % Pluronic F-127 (Sigma, #P2443) at room temperature for an overnight period. At day 20 of hiPSC-CM differentiation, the hiPSC-CMs were dissociated by using 0.25% Trypsin-EDTA and combined with hiPSC-cardiac fibroblast (hiPSC-CFs) in a 3:1 ratio at a density of 1×10⁷ cells/mL in a fibrin gel mixture containing 20 mg/mL fibrinogen from human plasma (Sigma, #F3879), RPMI/B27+ media with 100 U/mL thrombin from human plasma (Sigma, #605190). Each PDMS EHT was made with 100 μL of fibrin-cell suspension, which instantly polymerized at room temperature upon the addition of thrombin. The EHTs were cultured in RPMI/B27+ media supplemented with RI (10 μM), 20 g/mL aprotinin (Sigma-Aldrich #A3428) for 24 h after fabrication. The EHTs were then maintained for 10 days in RPMI/B27+ media containing 20 μg/mL aprotinin, with fresh media every 2 days for compaction. Following compaction (7-10 days post-fabrication), EHTs were treated with DMSO (control) and GANT61 (1.5 μM) for a period of 4 days. Following treatment, EHTs were analyzed for morphological analysis, contractility analysis, qPCR, and immunohistochemistry analysis.

RNA Isolation and qPCR Analysis

RNA isolation and qPCR analysis was performed using previously published protocols (Singh 2015). Briefly, hiPSC-CMs and EHTs were lysed in RNA-lysis buffer (Zymo Research, #R1057). Total RNA was extracted following the manufacturer's protocol. On-column DNA digestion was performed to eliminate genomic DNA as per the manufacturer's instructions. The RNA was eluted with DNase/RNase-free water and quantified using a NanoDrop spectrophotometer. For cDNA synthesis, 1 μg of total RNA was reverse transcribed by using the Superscript IV VILO kit (Thermo Fisher Scientific) according to the manufacturer's protocol in a 20 μl reaction volume. Quantitative PCR (qPCR) was performed using gene-specific primers and SYBR-green. For the qPCR (Roche Thermocycler, USA) analysis, each transcript was normalized to the housekeeping gene, EDF1. The list of primers used in this study is provided in the Table 1.

| S. No. | Gene | Forward (5′-3′) | Reverse (5′-3′) |
|---|---|---|---|
| 1 | EDF1 | ATCTTAGCGGCACAGAGACGAG | TGTCATGGTGCAGCTCCTCTGT |
| 2 | MYH6 | GGAAGACAAGGTCAACAGCCTGT | TCCAGTTTCCGCTTTGCTCGC |
| 3 | MYH7 | GGAGTTCACACGCCTCAAAGAGG | TCCTCAGCATCTGCCAGGTTGT |
| 4 | KCNJ2 | AACAGTGCAGGAGCCGCTTTGT | AGGACGAAAGCCAGGCAGAAGA |
| 5 | RYR2 | TCTTGAGGTTGGCTTTCTGCCAG | CTGTGCCAGCAAAGAGAGGAGCA |
| 6 | PLN | GCCTCAACAAGCACGTCAAAAGC | GCTGATGTGGCAAGCTGCAGATC |
| 7 | ATP2A2 | CCGGACTTTGAAGGCGTGGATTG | CCTCAGCAAGGACTGGTTTTCGG |
| 8 | PGC1α | CCCAAGGGTTCCCCATTTGAGA | TGGTTGGCTTTATGAGGAGGAGT |
| 9 | NRF1 | TCAGAATTGCCAACCACGGT | CGTCTGGATGGTCATCTCACC |
| 10 | MFN2 | TGGGCATTCTTGTTGTTGGAGGA | TGGTCCAGGTCAGACGCTCATA |
| 11 | MTND6 | CACCGTCACTCCTGAAGCC | TCTGTGCATCATGGATGCTACC |
| 12 | ACSL3 | TGCATACCATGGCTGCAGT | TCCTGTGGATCCACTTGTGTACA |
| 13 | CPT1A | ACGTCAATGGACAGCTACGC | TTCAGCGTTGAGGCCCATC |
| 14 | BAX | CAGGATGCGTCCACCAAGAAG | TGTCCACGGCGGCAATCATC |

### Bioinformatic Analysis

Using the inventors previously published bulk RNA-seq dataset (Singh 2015) (GEO GSE201589) to identify networks associated with a GLI-mediated mechanism in cardiomyocytes, the bioinformatic analysis workflow was performed as previously described with minor changes. We sorted the upregulated transcripts with log₂ fold change (log₂ FC>1.2) using R-studio package (v 4.3.1), in the GANT61 group versus controls and mapped the transcripts using the Entrez ID database (org.Mm.eg.db). These IDs were then uploaded to KEGG pathways and their second-level functional classes via KEGGREST. Pathways lacking class information were excluded. Data wrangling was carried out with openxlsx and the tidyverse, and heat maps were generated with ComplexHeatmap and circlize. We then isolated pathways classified under “Cellular Processes,” converted the corresponding gene-to-pathway pairs into a bipartite graph (using igraph), and rendered a hive plot in base R-package. For cluster analysis, transcripts with log₂ FC>0.8 were submitted to clusterProfiler's enrichKEGG; pathways with Benjamini-Hochberg-adjusted p<0.05 were retained. The 20 pathways with the highest gene ratios were ranked and visualized in ggplot2 as a size- and color-scaled dot plot. Data organization relied on openxlsx, dplyr; and stringr. To assess the mitochondrial- and metabolism-related pathways, upregulated transcripts with log₂ FC≥0.8 were used and Gene Ontology (GO) for Biological Process (BP) enrichment was performed with clusterProfiler's enrichGO. Significant terms following Benjamini-Hochberg correction were ranked by adjusted p-value. GO terms classified as mitochondrial or metabolic, were counted and expressed as a fraction of all enriched terms; these percentages were visualized as a two-slice pie chart with ggplot2 and ggforce. To further visualize the upregulated transcripts for their association with mitochondrial and metabolic GO terms, we extrapolated the relevant GO terms and their member genes and the combined set was converted to a bipartite edge list linking GO terms to their member genes and visualized as a gene concept network in igraph. GO nodes were color-coded, while all gene nodes were beige, and the network was presented in circular and Fruchterman-Reingold layouts. To ascertain further which genes were playing a role in the associated up-regulation of mitochondrial- and metabolic-related GO terms, gene node transcript log₂ FC values were compared using a two-column heat map generated with the ComplexHeatmap package.

Immunostaining of hiPSC-CM and EHTs

Immunostaining was performed as described previously (Hsieh 2022). Briefly, hiPSC-CMs or EHTs were washed with 1×DPBS and fixed in 4% paraformaldehyde solution for 15 min at RT. Fixed EHTs were processed sequentially in 5%, 15%, and 30% sucrose solution for 48 h, embedded into cryo-molds using OCT, and stored at −80° C. until cryo-sectioning. Using a cryostat (Leica CM1900), the EHTs were sectioned longitudinally and 8 m thickness sections were collected on a SuperPlus glass slide. For staining, cells or EHTs were washed with 1×PBS and permeabilized by incubation with 0.2% Triton X-100 for 45 min at RT. Samples were then washed twice with 1×PBS and blocked with BGST (50 g/L BSA, 10 g/L glycine, 2% Goat Serum, 0.1% Triton-X-100) solution for 2 h at room temperature. Primary antibodies against α-actinin (1:500; Abcam, #ab9465) and CX43 (1:200; Santa Cruz Biotech) diluted in BGST solution were applied overnight at 4° C. Following overnight incubation, samples were washed twice with 0.2% Tween-20 in PBS and once with 1×PBS. After that, samples were incubated with Alexa Fluor 488 (1:400; Invitrogen, #A21202) and Cy3 (1:400, Jackson Immunoresearch, #102649-700) tagged-secondary antibodies for 1.5 h at RT. Samples were washed twice with 0.2% Tween-20 in PBS and once with 1×PBS. Samples were incubated with 1 μg/μl DAPI for 10 min and washed with 1×PBS three times. Images were acquired at 40× magnification using an EVOS M5000 fluorescent microscope (Invitrogen USA) or Fluoview FV3000 confocal microscope (Olympus, USA).

### Flow Cytometry Analysis

hiPSC-MLC2v-EGFP lines were provided. hiPSC-MLC2v lines are differentiated as described previously (Garay 2022). Following differentiation and lactate enrichment, on day 20, hiPSC-MLC2V-GFP-CMs were replated and subsequently treated with GANT61 between D24-D26 and analyzed on D27. For analysis, hiPSC-MLC2V-GFP-CMs were washed with 1×DPBS, dissociated by using 0.25% trypsin for 10-12 min and collected into a 15 mL Falcon tube. Cells were centrifuged at 200×g for 5 min. The supernatant was discarded, and cells were washed with 1×DPBS, followed by centrifugation at 200×g for 5 min. The resulting cell pellets were resuspended in 500 μL of flow buffer containing 2 μg/ml propidium iodide (PI) and incubated for 10 min at RT in the dark. Samples were analyzed using a BD Biosciences flow cytometer.

### Contractility Analysis

On day 20 of differentiation, hiPSC-CMs were seeded in a Matrigel-coated 12-well plate. After 48 h of plating, hiPSC-CMs were treated with GANT61 (1.5 μM) for 48 h. Contractility was assessed by sequential phase-contrast imaging acquired at 10× magnification with 10 frames per second for 20 sec at 37° C. using a Leica microscope. The captured images were analyzed using ImageJ software and custom MATLAB script. Beat rate, contraction velocity, and relaxation velocity were plotted using GraphPad Prism Software.

### MorphoScript Analysis

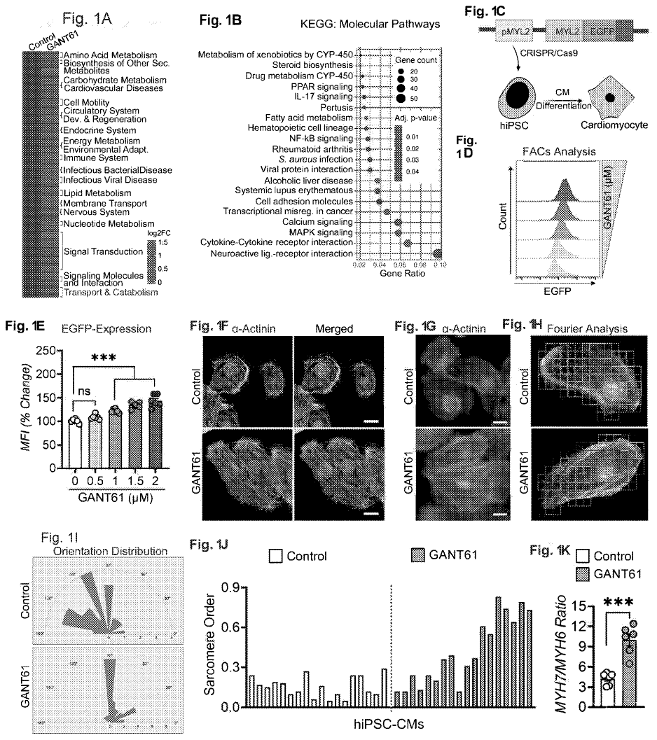

MorphoScript analysis was performed as described previously (Homan et al. 2021). A MATLAB script was used to quantify sarcomere order and dispersion in α-actinin-stained hiPSC-CMs images, captured at 40× magnification using an M5000 EVOS microscope (Invitrogen, USA). DAPI-stained images were first used to identify individual cells and distinguish them from clusters by outlining their boundaries. ImageJ post-processing was used to enhance the contrast of the images by 0.35% and then uploaded into MATLAB with a pixel-to-micron conversion factor of 0.227 μm. The area of each cell was manually selected and shaped as illustrated in FIG. 1G, H. Sarcomere order and dispersion were quantified automatically using the model with a window size of 15, an overlap of 0.5, and a detection threshold of 0.0065. To optimize the detection parameters, the threshold value was averaged from three cells showing clear striations and applied consistently across all cells analyzed.

### Patch-Clamp and Electrophysiology Analysis

On day 20 of differentiation, hiPSC-CMs were seeded onto Matrigel-coated glass coverslips for 48 h prior to treatment. Following treatment with GANT61 (1.5 μM) for 48 h, monolayer hiPSC-CMs were prepared for electrophysiological analysis. Coverslips were placed into a perfusion chamber mounted on an inverted microscope (Olympus BX51). Myocytes were continuously perfused with (in mM): 130 mM NaCl, 5.4 mM KCl, 1 mM CaCl2, 1 mM MgCl2, 5.5 mM D-Glucose, 5 mM HEPES, pH 7.4 (with NaOH) maintained at 37° C. Fire-polished borosilicate patch pipettes (3.5-4.5 MΩ) were filled with a K-Gluconate internal solution (in mM): 140 mM K-Gluconate, 2 mM MgCl2, 1.1 mM EGTA, 5 mM HEPES, 2 mM Na2-ATP, 0.3 mM Na-GTP, and 5 mM phosphocreatine, pH 7.2 (with KOH) After obtaining a stable whole cell configuration myocytes were held at −80 mV potential in current clamp mode and action potentials were evoked by a 1 Hz current injection (3 ms). Recordings were obtained with an EPC10 HEKA amplifier and Patchmaster 2×73.2 software (HEKA Elektronik). Action potential parameters were calculated with the AP analysis module in Patchmaster.

### Statistical Analysis

All the experiments and data were obtained from at least three independent biological experiments. Group comparisons were performed using one-way ANOVA with multiple comparisons test, and unpaired t-tests using the Mann-Whitney method, as appropriate. Statistical significance was defined as follows: *p<0.05; **p<0.01; ***p<0.001. All statistical analyses were conducted using GraphPad Prism version 9.3.1 (GraphPad Software, La Jolla, CA, USA). Error bars represent the standard error of the mean (SEM). The analyzed data are shown as mean±SEM.

### EXAMPLES

### Example 1: GLI Modulation Promotes a Maturation Program in Immature Cardiomyocytes

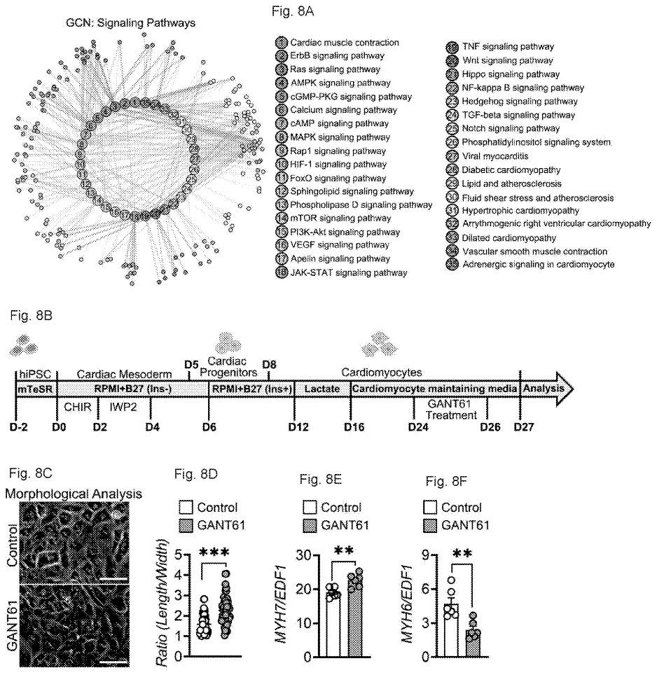

To examine the potential role of GLI signaling in CM development and maturation, we reanalyzed published RNA-seq datasets obtained from isolated neonatal murine CMs following treatment with HH/GLI inhibitor, GANT61 (pan-GLI inhibitor) for a 48 h period. Analysis was focused using the up-regulated transcripts and first examining the physiological functions that were enriched in this category. A heat map of the differentially upregulated transcripts (log₂ fold change >2) mapped to KEGG pathways for physiological functions (grouped by second-level physiological function categories), is shown in FIG. 1A. It was found that GLI modulation significantly enriched pathways associated with metabolic maturation, including carbohydrate, amino acid, lipid, and nucleotide metabolism. These findings are consistent with prior studies identifying metabolic remodeling as a central feature of hiPSC-CM maturation. Further, KEGG analysis for molecular pathways were mapped to steroid biosynthesis, fatty acid metabolism, Ca²⁺ signaling, cell adhesion and cell-receptor interactions (FIG. 1i). Collectively, these data provided a rationale to further investigate the role of GLI-signaling in CM maturation. Analysis of upregulated signal transduction pathways using GCN revealed enrichment of well-established cascades implicated in CM maturation, including Ca²⁺-MAPK, Hippo, JAK-STAT, and PI3K signaling. Many of these pathways including Ca²⁺-MAPK, Hippo, JAK-STAT, and PI3K are implicated in CM maturation (FIG. 8A). Additionally, we found an increase in transcripts related to calcium handling, adrenergic signaling, PPAR signaling and fatty acid metabolism (FIG. 1B). Together, these findings suggest that modulation of GLI signaling promotes metabolic and signaling features associated with CM maturation in immature neonatal murine CMs.

### Example 2: Modulation of GLI Signaling Improves Morphological, Structural, and Functional Maturation of hiPSC-CMs

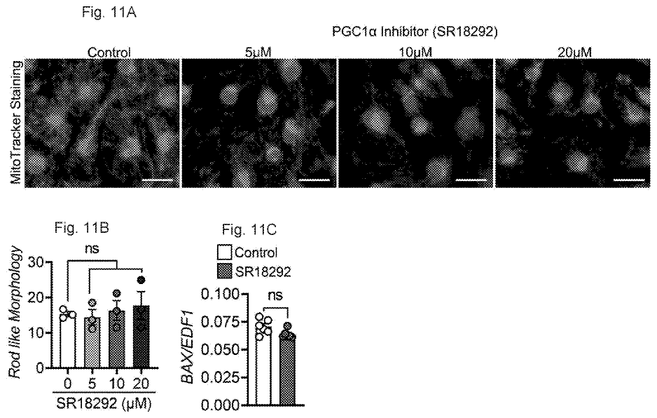

Based on the transcriptomic analysis, it was hypothesized that modulation of the GLI-signaling promotes CM maturation. The impact of GLI-modulation in CM maturation was directly tested using an MLC2v-EGFP reporter hiPSC-CM line, in which EGFP fluorescence correlates with ventricular CM maturation. This reporter line harbors an EGFP knock-in at the MLC2v locus, resulting in expression of an MLC2v-GFP fusion protein under the control of endogenous MLC2v (MYL2)-promoter (FIG. 1C). Flow cytometry analysis revealed a concentration-dependent increase in EGFP expression in the MLC2v-GFP-CM following treatment with GANT61 (0.5-2 μM) for a 48 h period (days 24-D26) relative to the controls (FIGS. 1D and 1E). Notably, the EGFP mean fluorescence reached a plateau by 1.5-2.0 μM and higher GANT61 concentration (>3.0 μM) induced CM stress (data not shown). Importantly, at 1.5-2.0 μM GANT61, no cell death was observed (data not shown). Based on these parameters, 1.5 μM was selected for the rest of the experimental design. To confirm these findings, an independent hiPSC line was differentiated to hiPSC-CMs and confocal microscopy performed following immunostaining for α-actinin to assess sarcomere organization, a hallmark of CM maturation. The analysis revealed that GANT61-treatment (1.5 μM, 48 hr) led to highly organized sarcomeric structures in hiPSC-CMs, whereas control hiPSC-CMs appeared rounded with poorly organized sarcomeres (FIG. 1F). Utilizing MorphoScript (a tool to analyze sarcomeric striations and their orientation) in these settings, analysis demonstrated that hiPSC-CMs treated with GANT61 displayed highly organized α-actinin striation and improved sarcomeric alignment (FIG. 1G-I), characterized by low dispersion (orientation distribution) and an increased order parameter (0.85 vs 0.28) (FIGS. 11 and 1J). Morphometric analysis further revealed an increased length-to-width ratio, consistent with enhanced cellular anisotropy (FIGS. 8C and 1D). Because sarcomeric maturation involves a fetal-to-adult myosin heavy chain isoform switch, the expression of myosin-isoforms was examined to visualize the shift from the fetal isoform α-MYOSIN HEAVY CHAIN (MYH6), to mature isoform, MYH7. GANT61 treatment resulted in an increased MYH7/MYH6 ratio, driven by increased MYH7 and reduced MYH6 expression relative to controls (FIG. 1K; FIGS. 8E and 8F). Taken together, these results demonstrate that modulation of GLI-signaling enhances morphological and structural maturation of hiPSC-CMs.

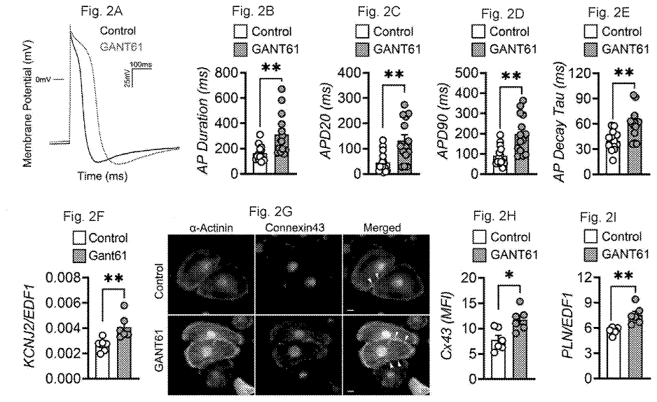

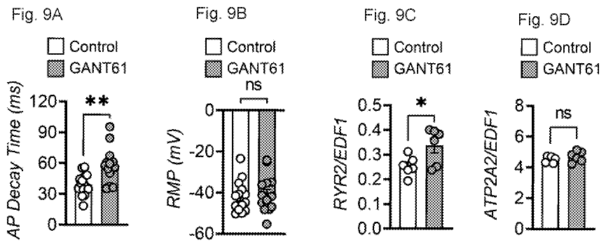

Functional maturation was then evaluated using whole-cell patch-clamp electrophysiology. GANT61-treated hiPSC-CMs exhibited significant prolongation of action potential duration at both 20% (APD20) and 90% (APD90) repolarization (FIG. 2A-D) relative to vehicle treated control. APD20 increased from 45.5±40.8 ms to 131.9±85.2 ms, while APD90 increased from 90.4±47.9 ms to 197.5±94.5 ms following GANT61 treatment (FIGS. 2C and 2D). These findings are in accordance with previous publications showing progressive increase in APD with time and maturation. In addition, we observed an increase in the AP decay tau and AP decay time in GANT61-treated hiPSC-CMs compared to control (FIG. 2E; 9 FIG. 9A) without any significant change in resting membrane potential (9 FIG. 9B). Consistent with these electrophysiological changes, qPCR analysis revealed increased expression of POTASSIUM INWARDLY RECTIFYING CHANNEL SUBFAMILY J MEMBER 2, (KCNJ2) upon GANT61 treatment (FIG. 2F). Next, immunostaining for gap junction protein, CONNEXIN 43 (CX43) showed enhanced punctate localization at cell-cell junctions in GANT61-treated hiPSC-CMs, indicative of improved electrical coupling (FIGS. 2G and 2H). Corroborating this, our qPCR analysis confirmed upregulation of transcripts involved in regulation of Ca²⁺ dynamics including PHOSPHOLAMBAN (PLN) and RYANODINE RECEPTOR 2 (RYR2), with no change in the ATPASE SARCOPLASMIC/ENDOPLASMIC RETICULUM CA²⁺ TRANSPORTING 2 (ATP2A2) expression in GANT61-treated hiPSC-CMs compared to control (FIG. 2I; FIGS. 9C and 2D). Overall, these results suggest that GLI-signaling modulation enhances the electrophysiological and functional maturation in hiPSC-CMs.

### Example 3: GLI Signaling Modulation Enhances Maturation in 3D-Engineered Heart Tissue

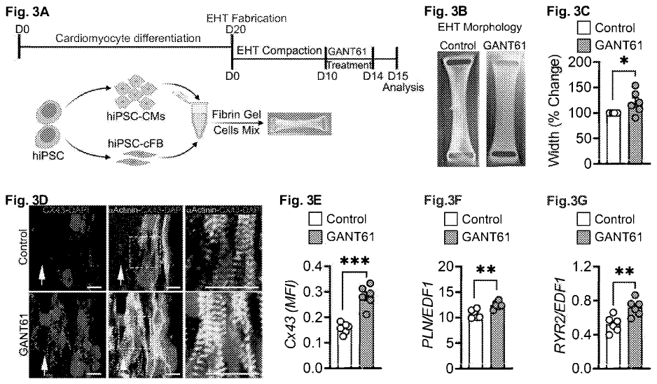

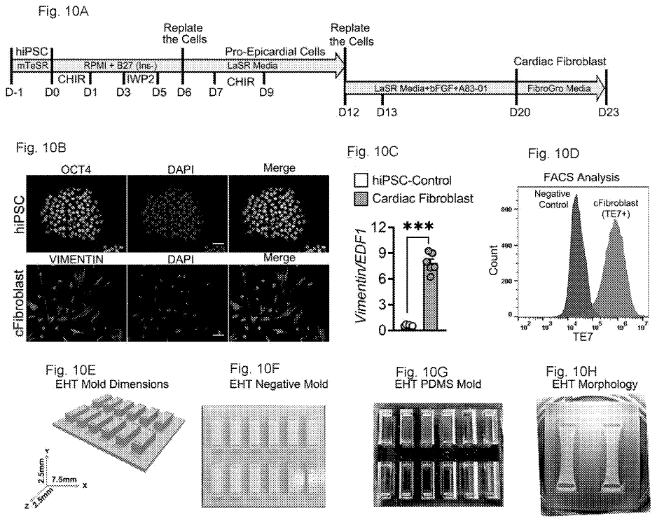

To assess whether GLI modulation promotes maturation in a more physiologically complex context, engineered heart tissue (EHT) was generated and GANT61-mediated maturation status was evaluated. First, optimized the hiPSC-derived cardiac fibroblast (hiPSC-CFs) differentiation protocol and obtained CFs with high vimentin expression and >95% TE-7⁺ (fibroblast marker) (FIG. 10A-10D). EHT constructs were fabricated by combining hiPSC-CMs and hiPSC-CFs at a 3:1 ratio in PDMS molds (FIG. 10E-10H), thereby approximating the cellular composition of the native myocardium (FIG. 3A). A detailed protocol for EHT-fabrication and maintenance is already published and described in the methods section. After 10 days of tissue compaction, EHTs were treated with GANT61 for 4 days and assayed for maturation kinetics (FIG. 3A). GANT61-treated EHTs exhibited improved tissue architecture with better tissue width relative to control EHT (FIGS. 3B and 3C).

To evaluate the cellular architecture, sarcomeric patterning and communications, immunostaining of EHT sections using α-actinin and CX43 antibodies was performed. Confocal microscopic examination revealed enhanced sarcomeric organization and preserved tissue integrity upon GANT61 treatment (FIG. 3D). Further, we found that the GANT61-treated EHTs showed more prominent CX43⁺ punctate dots at the cell membrane relative to the control tissues (FIGS. 3D and 3E). Consistent with these observations, our qPCR analysis showed increased expression of PLN and RYR2 upon GANT61 treatment compared to the control (FIGS. 3F and 3G). These results indicate that modulation of GLI-signaling enhances structural and functional maturation of EHTs, including improved CM alignment, intercellular coupling (via increased CX43 expression) and Ca²⁺ dynamics (via upregulation of PLN and RYR2). Based on these results, we hypothesize that GANT61 promoted CM maturation in the 3D-tissues.

### Example 4: Modulation of GLI Signaling Enhances Mitochondrial Dynamics in hiPSC-CMs

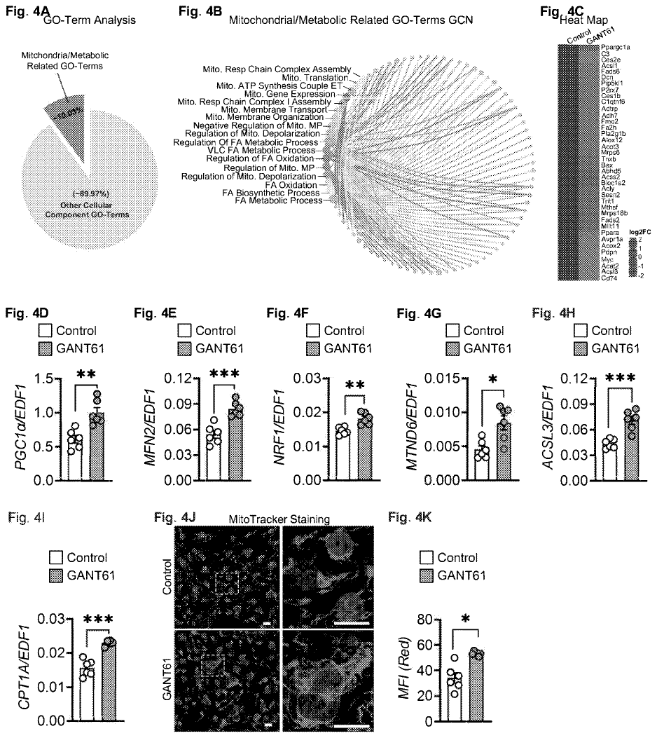

To elucidate mechanism underlying GLI-mediated CM maturation, a published dataset focusing on upregulated transcripts (log₂ fold change >0.8) was reanalyzed. Gene Ontology (GO)-analysis identified 5,339 significantly enriched cellular component terms. Notably, ~10% of GO-terms were associated with pathways involved in mitochondrial biogenesis and metabolic processes including oxidative phosphorylation, TCA cycle, ATP synthesis, mitochondrial expression, fatty acid oxidation (FAO), and regulation of mitochondrial membrane potential (FIGS. 4A and 4B). Furthermore, among all enriched categories, GCN analysis using the most abundant GO-terms showed strong connectivity between mitochondrial functions and FAO-related pathways (FIG. 4B). This is consistent with the metabolic transition toward oxidative metabolism characteristic of CM maturation. Recognizing this, a detailed mitochondrial network visualization was performed and identified, PEROXISOME PROLIFERATOR-ACTIVATED RECEPTOR GAMMA, COACTIVATOR 1 ALPHA (PPARGC1A; PGC1α) as the highly enriched and interconnected node linking mitochondrial and metabolic pathways (FIG. 4C). PGC1α is known as a master regulator of mitochondrial biogenesis, function, and energetics. Based on results and published findings, it is proposed that GANT61 mediates CM maturation through engagement of PGC1α-associated mitochondrial programs. Indeed, qPCR analysis showed induced expression of PGC1α together with increased expression of its downstream effectors, including MITOFUSIN 2 (MFN2), and NUCLEAR RESPIRATORY FACTOR 1 (NRF1) following GANT61 treatment (FIG. 4D-F), along with elevated expression of mitochondrial gene, MITOCHONDRIAL DNA-ENCODED NADH DEHYDROGENASE SUBUNIT 6 (MTND6) as compared to the control group (FIG. 4G). Next, evaluation of the factors involved in FAO showed higher levels of ACYL-COA SYNTHETASE LONG-CHAIN FAMILY MEMBER 3 (ACSL3) and CARNITINE PALMITOYL TRANSFERASE IA (CPT1A) transcripts (FIGS. 4H and 4I). These findings support the notion that GANT61 treatment leads to improved mitochondrial biogenesis and function with enhanced FAO. To ascertain this, we performed live-cell imaging following staining of hiPSC-CMs with MitoTracker-Red fluorescent dye to visualize the mitochondrial network. Our analysis showed that modulation of GLI-signaling enhanced the mitochondrial density and network complexity in GANT61-treated hiPSC-CMs (FIGS. 4J and 4K). Together, these results demonstrate that modulation of GLI-signal promotes CM maturation via the activation of mitochondrial dynamics and metabolic maturation.

### Example 5: GLI-Signaling Induces CM Maturation Via the PGC1α-Axis

Having established the positive correlation of GANT61-mediated induction of PGC1α and its downstream effectors, experiments were set up to test this axis as a plausible mechanism during the CM maturation process. To directly test the role of the GLI-PGC1α axis, we utilized SR18292, a small molecule inhibitor of PGC1α, to block mitochondrial biogenesis and function. We treated the hiPSC-CMs with increasing concentrations of SR18292 (5-20 μM) for 48 h and assayed for mitochondrial dynamics following staining using MitoTracker-Red fluorescent dye and live-cell imaging. Our data showed that the mitochondrial density and network were reduced in the SR18292-treated hiPSC-CMs in a concentration-dependent manner (FIG. 11A). These data further supported the critical role of PGC1α in the regulation of mitochondrial biogenesis. In these settings, we did not observe any morphological changes in hiPSC-CM (FIG. 11B). Further, analysis showed no change in cell death or contractile behavior in these conditions (data not shown). qPCR analysis did not show any change in the BAX gene expression following SR18292-treatment (FIG. 11C). Based on these data, we selected 10 μM as an optimal concentration to block PGC1α activity for subsequent experiments.

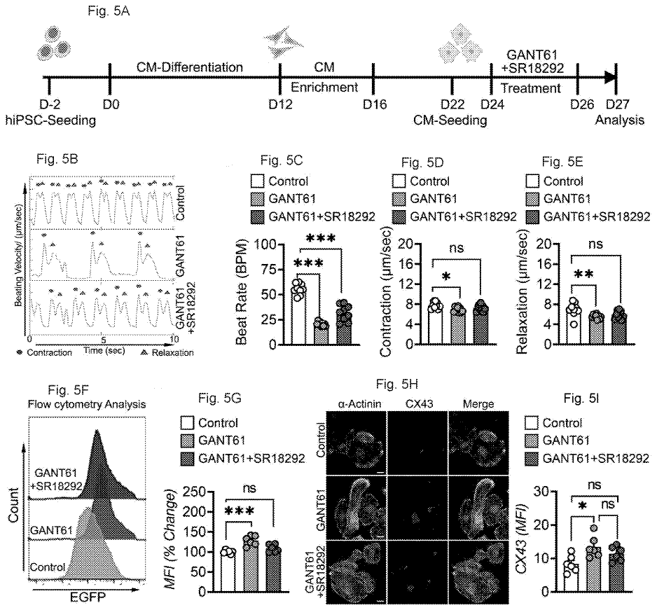

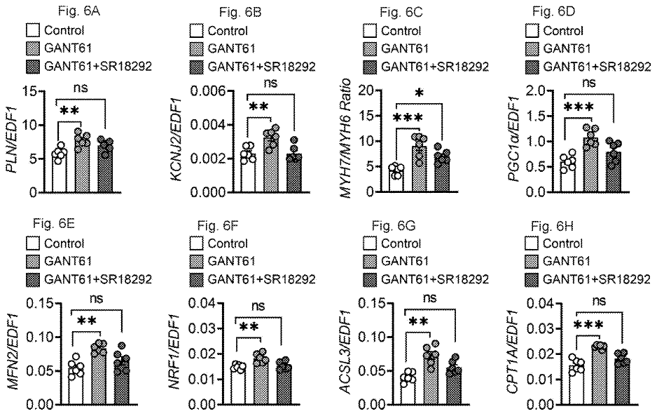

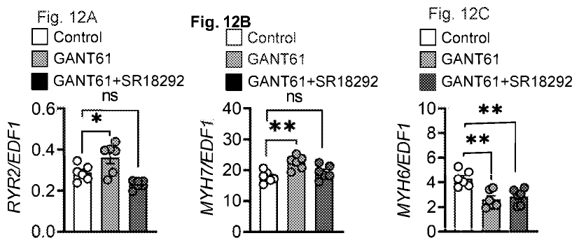

To define the GLI-PGC1α mediated mechanism, contractility analysis was performed upon GANT61 treatment in the presence or absence of PGC1α inhibitor (SR18292) (FIG. 5A). As indicated above, we found significantly reduced beat rate following GANT61 treatment alone (FIGS. 5B and 5C). Whereas a combinatorial treatment of hiPSC-CMs with GANT61 and SR18292 (GANT61+SR18292) led to a significant increase in the beat rate relative to the GANT61-only conditions (FIGS. 5B and 5C). We did not find any change in the contraction speed and relaxation speed in the GANT61+SR18292 condition relative to the control condition (FIGS. 5D and 5E). It is shown that immature CMs have a higher beat rate as compared to the mature CMs. Since improved beat rate in the GANT61+SR18292 conditions was observed, it was proposed that mitochondrial pathways act downstream of GLI-signaling and play a critical role in GANT61-mediated modulation of contractile maturation of hiPSC-CMs. Next, utilizing MLC2v-EGFP-CM and performing flow-cytometry analysis for EGFP expression in GANT61+SR18292 condition, it was found that while GANT61-treatment led to a higher EGFP expression, whereas, GANT61+SR18292 suppressed the EGFP levels similar to the control groups (FIGS. 5F and 5G). Next, confocal microscopy of the α-actinin stained hiPSC-CMs showed highly organized sarcomeres in GANT61 conditions but were less prominent in the GANT61+SR18292 condition relative to control (FIG. 5H). To further investigate this axis, immunofluorescence analysis was performed for the Gap junction protein CX43, in these conditions. While GANT61 alone significantly increased the CX43 expression relative to the control, the levels of CX43 were similar to control in the GANT61+SR18292 condition (FIGS. 5H and 5I). Overall, these results indicate that PGC1α activity is required for GLI-modulation-induced hiPSC-CM maturation. To further define the GLI-PGC1α axis in mediating CM maturation, qPCR analysis for genes involved in Ca²⁺ handling showed induced expression of PLN, KCNJ2, and RYR2, in GANT61 conditions, whereas, the levels of these transcripts were similar to that of control following GANT61+SR18292 treatment (FIGS. 6A and 6B, FIG. 12A). Further evaluation of myosin isoform expression showed that while MYH6 levels were reduced in both treatment groups, MYH7 expression remained comparable to control in the GANT61+SR18292 condition, whereas it was significantly increased following GANT61 treatment alone (FIG. 6C; FIGS. 12B and 12C). We next examined genes involved in mitochondrial biogenesis and fatty acid oxidation (FAO). qPCR analysis demonstrated that GANT61 treatment alone significantly increased expression of PGC1α, MFN2, NRF1, ACSL3, and CPT1A, whereas co-treatment with SR18292 reduced their expression to levels comparable to control (FIG. 6D-H). Collectively, these findings demonstrate that PGC1α activity is required for GLI-modulation-induced structural and metabolic maturation of hiPSC-CMs, supporting a functional GLI-PGC1α mediated program in this process.

What is claimed is:

1. A method for promoting maturation of cardiomyocytes, comprising contacting cardiomyocytes with an agent that modulates GLI signaling, wherein the modulation of GLI signaling induces maturation of the cardiomyocytes, wherein in the cardiomyocytes are human induced pluripotent stem cell-derived cardiomyocytes (hiPSC-CMs).

2. The method of claim 1, wherein the modulation of GLI signaling comprises inhibition of one or more GLI transcription factors selected from GLI1, GLI2, and GLI3.

3. The method of claim 1, wherein the agent is a small molecule inhibitor of GLI signaling comprising GANT61.

4. The method of claim 1, wherein the hiPSC-CMs are cultured in a two-dimensional monolayer culture or comprise three-dimensional engineered cardiac tissue.

5. The method of claim 1, wherein hiPSC-CMs are contacted with the agent for about 24 hours to about 96 hours.

6. The method of claim 1, wherein the agent is administered at a concentration of about 0.5 μM to about 2 μM.

7. The method of claim 1, wherein maturation of the cardiomyocytes comprises increasing expression of cardiomyocyte maturation markers. selected from MYH7, MYL2, KCNJ2, PLN, RYR2, and CX43.

8. The method of claim 1, wherein the mature cardiomyocytes exhibit enhanced electrophysiological properties as compared to mature cardiomyocytes of a different agent that modulates GLI signaling.

9. The method of claim 1, wherein the mature cardiomyocytes exhibit improved calcium handling, reduced spontaneous beat rate, increased contractile force generation or combinations thereof as compared to mature cardiomyocytes of a different agent that modulates GLI signaling.

10. The method of claim 1, wherein modulation of GLI signaling activates a PGC1α regulatory pathway.

11. The method of claim 10, wherein activation of the PGC1α pathway increases expression of genes involved in mitochondrial biogenesis and the gene comprises NRF1, MFN1, MFN2, CPT1A, or ACSL3.

12. A method for inducing maturation of human induced pluripotent stem cell-derived cardiomyocytes, comprising:
contacting the cardiomyocytes with GANT61 in culture, wherein the GANT61 inhibits GLI signaling and thereby promotes structural, electrophysiological, and metabolic maturation of the cardiomyocytes.

13. The method of claim 12, wherein the cardiomyocytes exhibit increased sarcomere alignment following contact with GANT61 for a time period ranging from 24 hours to 96 hours.

14. The method of claim 12, wherein the cardiomyocytes exhibit increased expression of MYH7 relative to MYH6 following contact with GANT61 for a time period ranging from 24 hours to 96 hours.

15. A cardiomyocyte maturation culture system, comprising
cardiomyocytes derived from pluripotent stem cells; and
a GLI signaling modulator present in culture medium in an amount sufficient to promote cardiomyocyte maturation at a concentration of 0.5 μM to 2.0 μM,
wherein the GLI signaling modulator is capable of activating a PGC1α-mediated metabolic maturation pathway.

16. The system of claim 15, wherein the GLI signaling modulator is GANT61.

17. The system of claim 15 wherein the cardiomyocytes derived from pluripotent stem cells are human induced pluripotent stem cell-derived cardiomyocytes (hiPSC-CMs) cultured in a 2 dimensional monolayer.

18. The system of claim 15 wherein the cardiomyocytes derived from pluripotent stem cells are human induced pluripotent stem cell-derived cardiomyocytes (hiPSC-CMs) are 3 dimensional engineered heart tissue (3D-EHT).
